(19) United States
(12) Patent Application Publication
(10) Pub. No.: US 2014/0155855 A1
(43) Pub. Date: Jun. 5, 2014

(54) Adjustable Pant-Like Disposable Undergarment With Attachment Assembly Concealing A Line Of Weakness And Method Of Manufacture

(71) Applicant: KIMBERLY-CLARK WORLDWIDE, INC., Neenah, WI (US)

(72) Inventors: Chadwick Irvin Romzek, Neenah, WI (US); Brian Keith Rhodes, Larsen, WI (US); Jason Andrew Verboomen, Appleton, WI (US); Bradley William Schoon, Oshkosh, WI (US)

(73) Assignee: Kimberly-Clark Worldwide, Inc., Neenah, WI (US)

(21) Appl. No.: 13/690,534

(22) Filed: Nov. 30, 2012

(51) Int. Cl. A61F 13/15; A61F 13/49

(52) U.S. Cl. CPC A61F 13/15577; A61F 13/49007; A61F 13/15723
USPC 604/385.11; 156/66

(57) ABSTRACT

A method and apparatus for producing a pre-fastened adjustable pant-like disposable absorbent undergarment including a front body panel having a line of weakness is disclosed. The method includes providing a moving web having a waist end and a leg-cut-out end. The moving web is weakened to form a line of weakness defining a leading portion of the moving web and a trailing portion of the moving web at the line of weakness. Desirably, the line of weakness includes a series of weakened areas having a combined length in a cross-machine direction at least 85% of a distance between the waist end and the leg cut-out end. The method further includes passing it through a fastener attachment station to bridge the leading portion and the trailing portion of the moving web together with a fastener assembly.

### BACKGROUND

Pant-like disposable undergarments for absorbing human discharges can appear similar in size and shape to regular cloth underwear which is designed to be laundered and reused. A disposable absorbent undergarment is intended to be worn by persons, including infants, toddlers, or adults, and is designed for a single or temporary use and is meant to be disposed of after being used once instead of being laundered or dry cleaned for re-use. Some examples of disposable undergarments include infant diapers, training pants, adult incontinence garments, feminine pants, etc.

Some pant-like disposable absorbent undergarments manufactured today resemble regular cloth underwear in that they have a waist opening and a pair of leg openings. Such pant-like disposable absorbent undergarments can be pulled up around the torso of a wearer in a similar fashion as regular cloth underwear. Still other pant-like disposable absorbent undergarments have an open or flat configuration and are designed to be placed adjacent to a wearer's torso and then rely upon one or more attachment tabs or fasteners to secure the undergarment around the wearer's torso. This design is beneficial for bed bound users who may be immobile or for children who need assistance in securing the undergarment in place. Still other adjustable, pant-like absorbent undergarments contain attachment means for opening and closing the waist opening after the undergarment has been positioned around the wearer's torso. This type of adjustable undergarment has an advantage in that the wearer does not have to remove outer clothing in order to check the status of the undergarment or to remove the undergarment from their body.

One example of such an adjustable, pant-like disposable absorbent undergarment includes a pair of lines of weakness that a user must break to enable adjusting the fit of the undergarment. The lines of weakness usually extend from the waist opening to one of the leg openings and are designed to be broken either prior to positioning the undergarment around the user's torso or while the undergarment is already positioned around the wearer's torso. Fastener assemblies are then utilized to refasten the undergarment so that it is snug about the wearer's torso. Often, each line of weakness may be ergonomically hard to tear open by older adults, some of who may be suffering from arthritis or other medical ailments.

Typically, the types of consumer goods mentioned above are manufactured on a continuous basis on large scale manufacturing lines. Usually, various raw products or components are formed on, or integrated into, a continuous stream of material, which often includes a web of material that moves in a machine direction through and along the line. As such, it is important to maintain the integrity of the stream of material or web during the process so as to avoid costly downtime. In general, the web is pushed or pulled along the line, so as to put the web in tension. Accordingly, the formation of a partially severed panel, especially along a cross-direction, can increase the risk of continuous web breakage. Therefore, it is desirable to maintain the tensile strength of the stream of materials or web as it passes through the process.

Web panel stability is an important aspect to maintaining the integrity of the process. The stability of the moving web can be maintained with use of the stability control module while conveying the moving web before the cutting station until after the fastener assembly is attached at the fastener assembly attachment station. The stability control module may include vacuum, surface roughness and/or needle/clamp engagement or other suitable means known to those skilled in the art to ensure stability of the web on the conveying means.

An adjustable, pant-like disposable absorbent undergarment that includes a panel having a more easily broken line of weakness is needed to enable the line of weakness to be broken in the manufacturing process or more easily by the consumer. Additionally, there is need to provide a method and apparatus for manufacturing a pant-like disposable absorbent undergarment that includes a front panel with a line of weakness without causing a problem with the integrity of the stream of material or web during the process.

### SUMMARY

Generally, a method and apparatus for producing a pre-fastened adjustable pant-like disposable absorbent undergarment, including a front body panel having a line of weakness, is disclosed. The method includes providing a moving web having a waist end and a leg-cut-out end. The moving web is weakened to form a line of weakness defining a leading portion of the moving web and a trailing portion of the moving web at the line of weakness. Desirably, the line of weakness includes a series of weakened areas having a combined length in a cross-machine direction at least 85% of a distance between the waist end and the leg cut-out end to enable easier opening of the undergarment. The method further includes passing it through a fastener attachment station to bridge the leading portion and the trailing portion of the moving web together with a fastener assembly.

The line of weakness may have a variety of configurations. In one embodiment, the series of weakened areas having a combined length in a cross-machine direction at least 90% of a distance between the waist end and the leg cut-out end. In another embodiment, the series of weakened areas having a combined length in a cross-machine direction at least 99% of a distance between the waist end and the leg cut-out end.

In another example, the line of weakness may include a first region at the waist end having a first perforation pattern and a second region at the leg cut-out end having a second perforation pattern. The line of weakness further may include a third region extending between the first region and second region, the third region having a third perforation pattern. The third perforation pattern may be different than the first and second perforation pattern.

In one embodiment, the first and second perforation pattern may include a first perforation length and the third perforation pattern may include a second perforation length, wherein the first perforation length is greater than the second perforation length. Having a longer perforation length at the waist end and leg cut-out end may allow for easier breaking of the line of weakness on the product. In one particular embodiment, the first perforation length may be greater than about 10 mm. Alternatively, the first perforation length is less than the second perforation length. Having a shorter perforation length at the waist end and leg cut-out end may allow for easier breakage of the line of weakness on the product.

In another embodiment, the line of weakness may further include a fourth region in a middle portion of the body panel, the fourth region may have at least one perforation extending between 10% and 85% of the distance between the waist end and the leg cut-out end. This fourth region may also include a plurality of perforations.

In another embodiment, the line of weakness may further include a fifth region extending between the fourth region and the third region. The fifth region may have a fourth perforation pattern. This fourth perforation pattern may have a perforation length of less than about 2 mm.

Alternatively, the first perforation length is less than the second perforation length. Having a shorter perforation length at the waist end and leg cut-out end may allow for easier breakage of the line of weakness on the product.

These features will be described in greater detail herein. Further, it is to be understood that both the foregoing general description and the following detailed description are exemplary and are intended to provide further explanation of the invention claimed.

### BRIEF DESCRIPTION

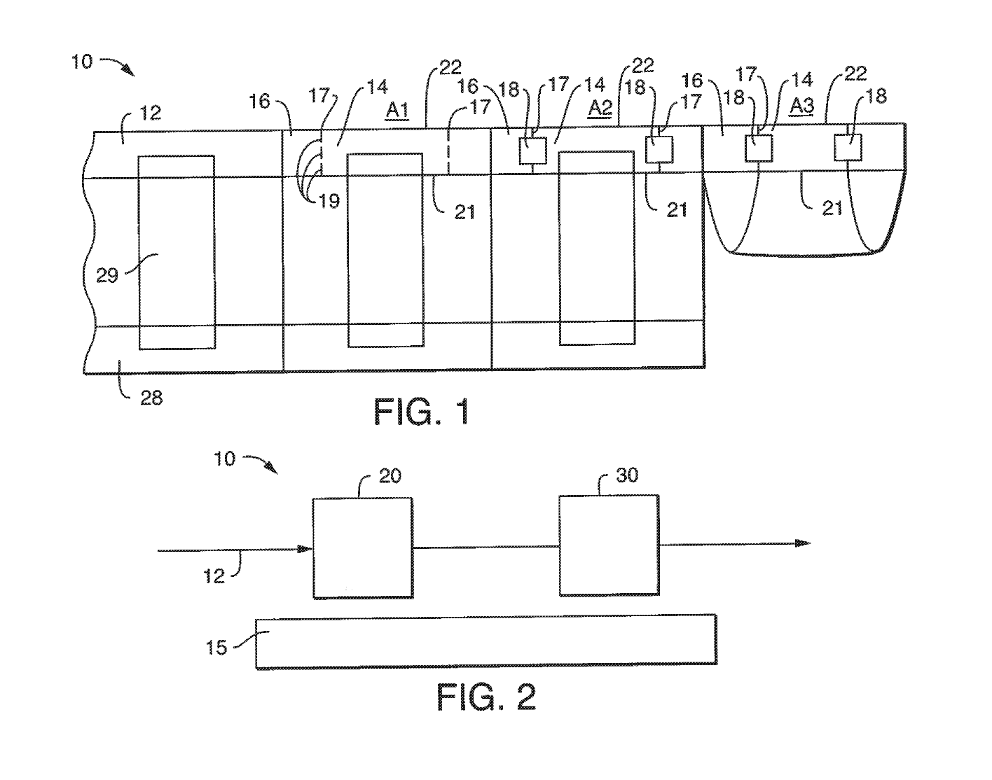

FIG. 1 depicts a method of producing a pre-fastened adjustable pant-like disposable absorbent undergarment including a front body panel having a line of weakness.

FIG. 2 depicts an apparatus for producing a pre-fastened adjustable pant-like disposable absorbent undergarment including a front body panel having a line of weakness.

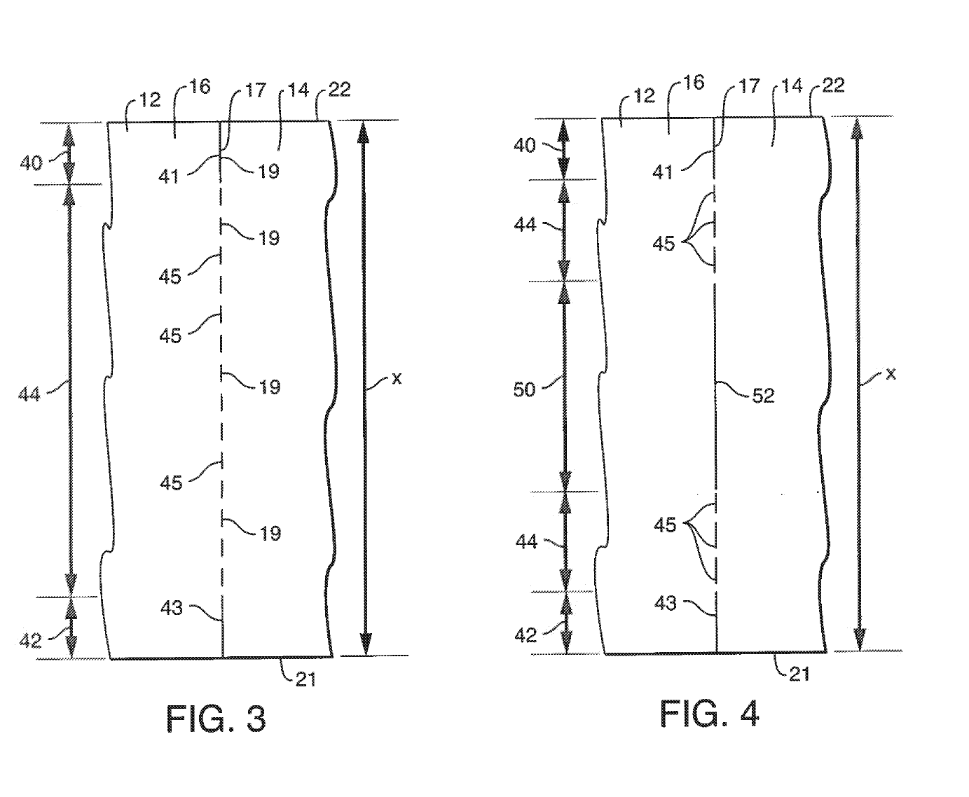

FIG. 3 illustrates one exemplary line of weakness for use on the pre-fastened adjustable pant-like disposable absorbent undergarment.

FIG. 4 illustrates another exemplary line of weakness for use on the pre-fastened adjustable pant-like disposable absorbent undergarment.

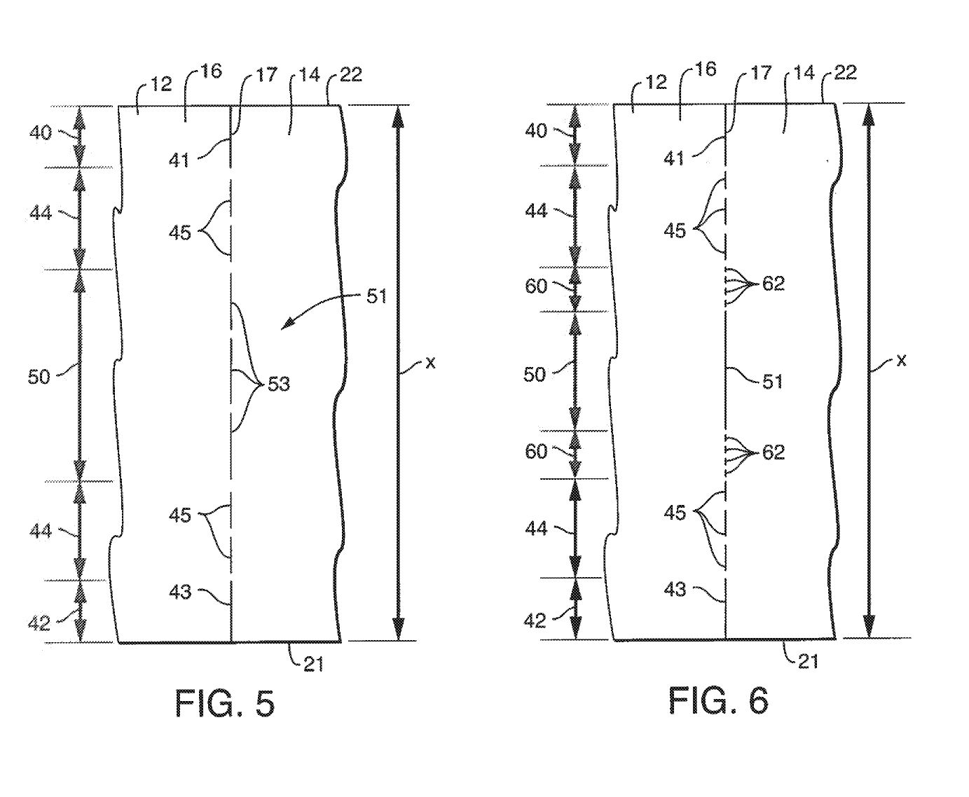

FIG. 5 illustrates another exemplary line of weakness for use on the pre-fastened adjustable pant-like disposable absorbent undergarment.

FIG. 6 illustrates another exemplary line of weakness for use on the pre-fastened adjustable pant-like disposable absorbent undergarment.

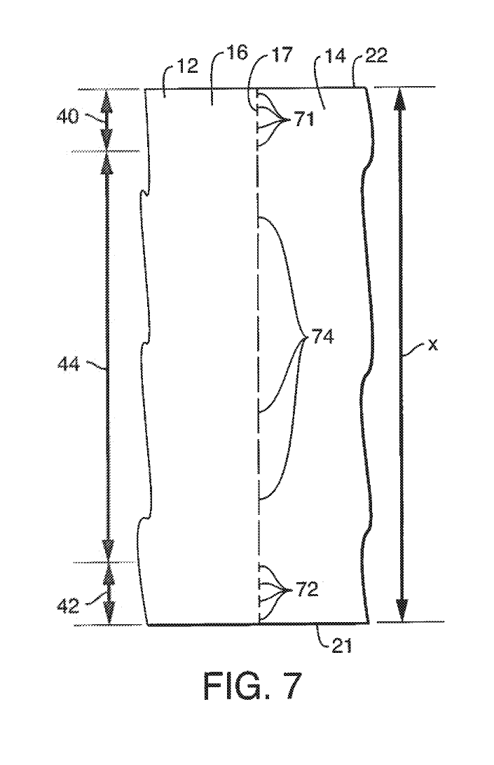

FIG. 7 illustrates another exemplary line of weakness for use on the pre-fastened adjustable pant-like disposable absorbent undergarment.

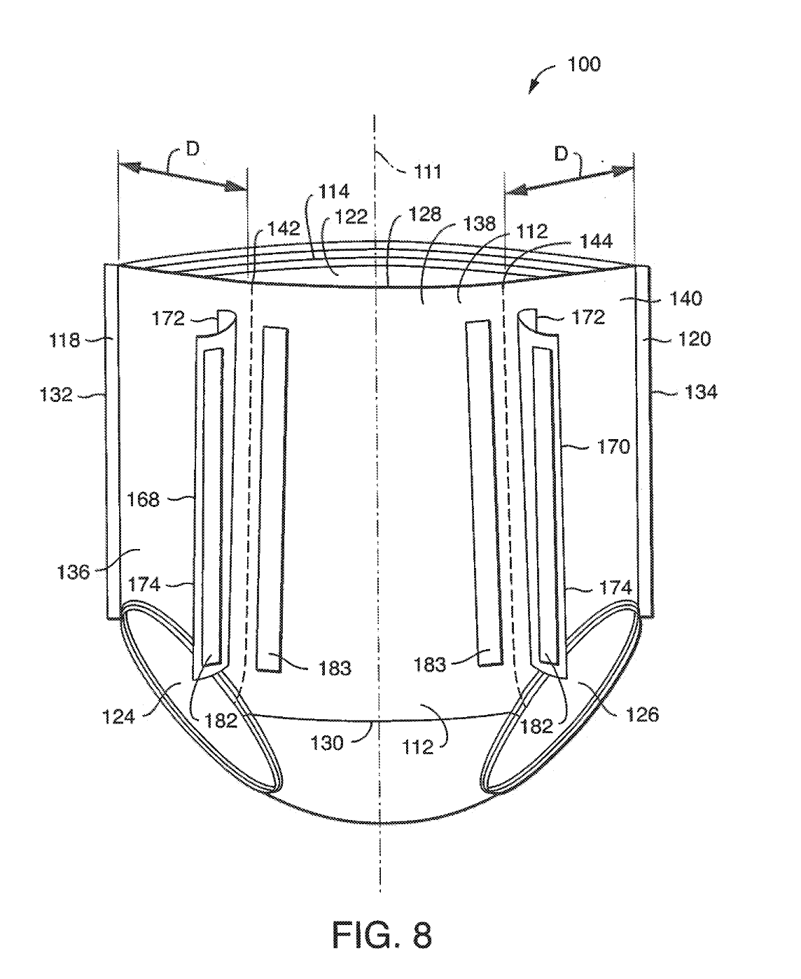

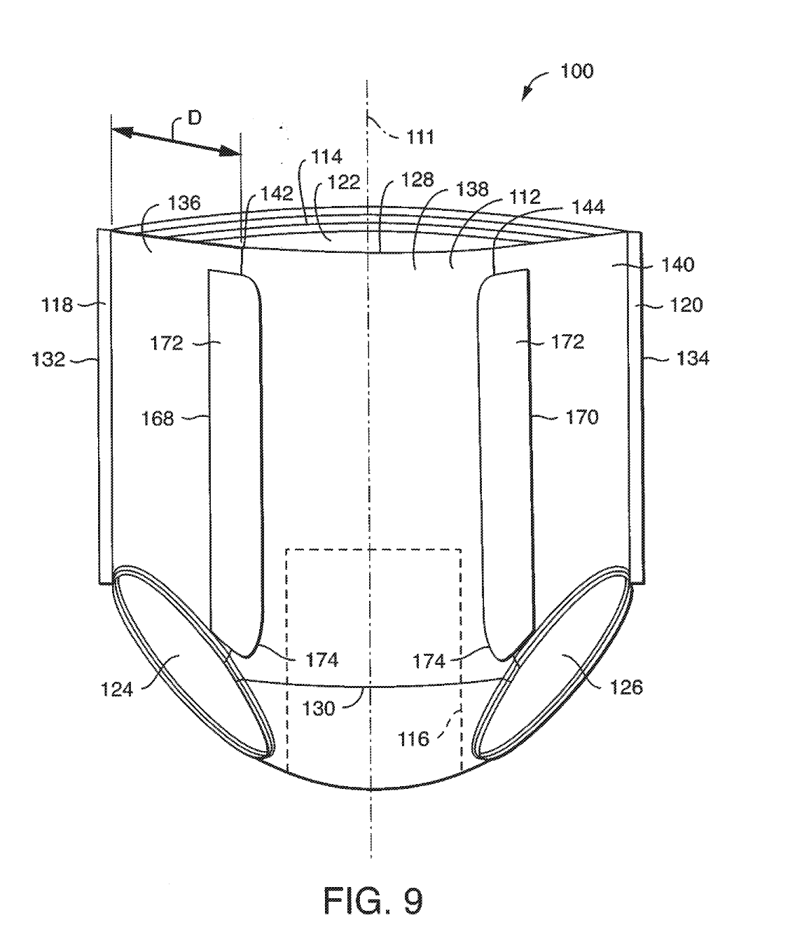

FIG. 8 depicts a front perspective view of one example of a garment, the garment shown in an unfastened, pant-like configuration.

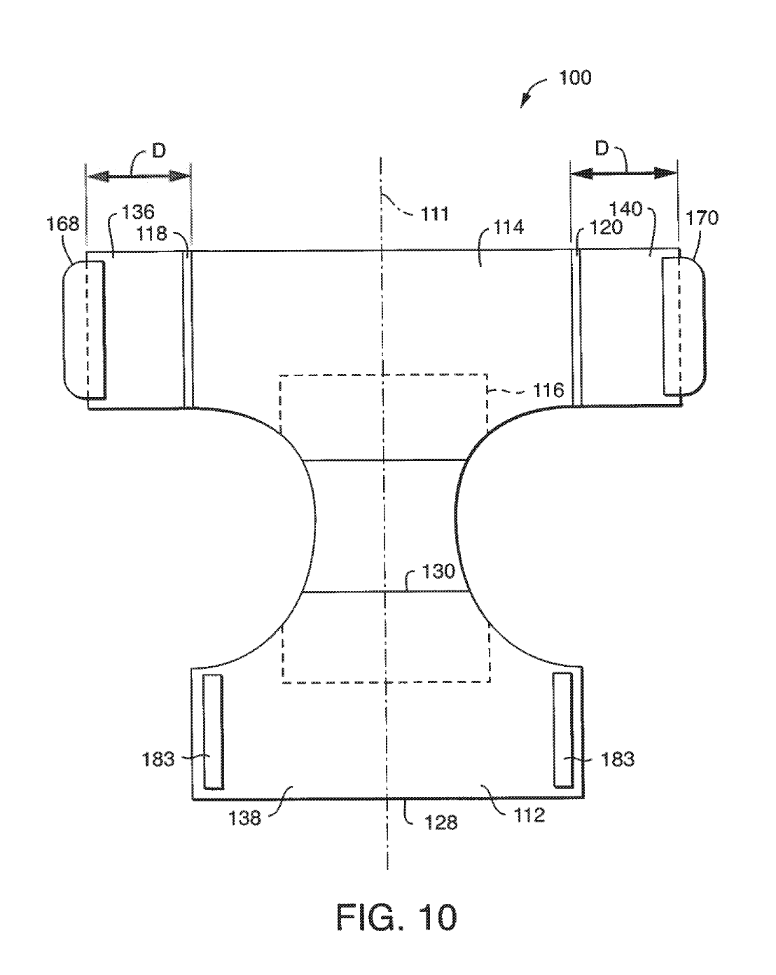

FIG. 9 depicts a front perspective view of one example of a garment, the garment shown in a pre-fastened, pant-like configuration.

FIG. 10 depicts a plan view of the garment of FIG. 8, the garment shown in an unfastened, laid-open, relaxed configuration.

### DETAILED DESCRIPTION

Reference to the Figures shall be made in describing various embodiments. It should be noted that the embodiments depicted in the Figures and described herein are merely representative examples. The various embodiments are suitable for use in conjunction with disposable absorbent undergarments such as refastenable adult incontinence underwear, pre-fastened disposable diapers, refastenable disposable training pants or swim pants, refastenable disposable enuresis garments, and the like. For illustration purposes, various embodiments shall be described in conjunction with refastenable incontinence or enuresis underwear.

Within the context of this specification, each term or phrase below will include the following meaning or meanings.

The term “body side” should not be interpreted to mean in contact with the body of the user, but rather simply means the side that would face toward the body of the user when the garment is applied to the user, regardless of whether the absorbent garment is actually being worn by the user and regardless of whether there are or may be intervening layers between the component and the body of the user Likewise, the term “garment side” should not be interpreted to mean in contact with the garments of the user, but rather simply means the side that faces away from the body of the user when the garment is applied to the user, and therefore toward any outer garments that may be worn by the user, regardless of whether the absorbent garment is actually being worn by a user, regardless of whether any such outer garments are actually worn and regardless of whether there may be intervening layers between the component and any outer garment.

The term “machine direction” means the direction of flow as the various members and webs progress along the fabrication line and process. It should be understood that various separate members or webs can each be traveling in a machine direction, but with the various machine directions not necessarily being parallel or oriented in the same direction. For example, one web may be traveling along a first machine direction, which is substantially perpendicular to the travel of another web in a second machine direction.

The term “cross direction” means the direction substantially perpendicular to the machine direction.

The term “downstream” means that one item is positioned more closely to the output or finished product end of the machine and/or process relative to another item. Conversely, the term “upstream” means that an item is positioned more closely to the input end of the machine or process relative to another item. For example, the output end is downstream of the input end, and vice versa, the input end is upstream of the output end.

The term “disposable absorbent undergarment” as used herein is an article that is intended to be worn by persons, including infants, toddlers or adults, which is designed for a single or temporary use and is meant to be disposed of after being used once instead of being laundered or dry cleaned for re-use.

The term “attached” refers to the joining, adhering, bonding, connecting, or the like, of two elements. Two elements will be considered to be attached together when they are attached directly to one another or indirectly to one another, such as when each is directly attached to intermediate elements.

The term “disposable” refers to articles which are designed to be discarded after a limited use rather than being laundered or otherwise restored for reuse.

The term “elastomeric” refers to a material or composite which can be elongated by at least 50% of its relaxed length and which will recover, upon release of the applied force, at least 20% of its elongation. It is generally preferred that the elastomeric material or composite be capable of being elongated by at least 100%, more preferably by at least 200%, of its relaxed length and recover, upon release of an applied force, at least 50% of its elongation.

The term “weakening” means to cause to lose strength, such that the area that is weakened is not as strong as the adjacent areas. For example, and without limitation, an area that is weakened may have a lesser tear or tensile strength as compared with the adjacent areas of the web, such that the web is more likely to be torn or broken along the area of weakness rather than the adjacent areas. In this way, the manufacturer can control the area of the web that will be broken, whether such breakage is performed by the end user or at a later time during the manufacturing or fabrication process.

The term “line of weakness” refers to any region or area of weakened material, preferably having a length and which may or may not have a defined width, and can include linear and non-linear patterns, such as curvilinear patterns of weakness, or other shapes, such as circles, rectangles, etc. The line of weakness can include a perforation or other series of cuts, a thinning such as scoring, or breakage or separation of material, or a strip of a different kind of material bridging between adjacent portions of material, that is more easily torn or broken than the adjacent portions, and which allow the user or manufacturer to separate the adjacent portions along the line of weakness.

The terms “longitudinal” and “transverse” have their customary meaning, as indicated by the longitudinal and transverse axes depicted in the Figures. The longitudinal axis lies in the plane of the article and is generally parallel to a vertical plane that bisects a standing wearer into left and right body halves when the article is worn. The transverse axis lies in the plane of the article generally perpendicular to the longitudinal axis.

These terms may be defined with additional language in the remaining portions of the specification.

A method and apparatus for producing a pre-fastened adjustable pant-like disposable absorbent undergarment including a front body panel having a line of weakness is disclosed. The method includes providing a moving web having a waist end and a leg-cut-out end. The moving web is weakened to form a line of weakness defining a leading portion of the moving web and a trailing portion of the moving web at the line of weakness. Desirably, the line of weakness includes a series of weakened areas having a combined length in a cross-machine direction at least 85% of a distance between the waist end and the leg cut-out end to enable easier opening of the undergarment. The method further includes passing it through a fastener attachment station to bridge the leading portion and the trailing portion of the moving web together with a fastener assembly.

Referring to FIGS. 1 and 2, a method and apparatus for producing a pre-fastened adjustable pant-like disposable absorbent undergarment is illustrated. A moving web 12 is shown as moving in a machine direction along a process line. The moving web 12 may be used as a front body panel, a back body panel, or both in the disposable absorbent undergarment. In some embodiments, the front body panel may include an integral or separate waistband.

The moving web 12 can be formed from a single piece of material or can be formed as a laminate consisting of two or more layers. The layers of the laminate can be of the same material or different material. In one embodiment, a laminate is formed from a first layer and a second layer. Sandwiched between the first and second layers are two or more elastic strands. Desirably, from two to about a hundred elastic strands can be utilized in the moving web 12 depending upon the overall size of each panel. The elastic strands can be formed from LYCRA, or a similar material. LYCRA is a trademark of INVISTA (Wichita, Kans.). The diameter and/or cross-sectional configuration of the elastic strands, the decitex (weight in grams per 10,000 meters) of the elastic strands, and the tension imparted into the elastic strands can all be varied to suit one's particular product needs. The exact number of elastic strands that are utilized should be sufficient to ensure that the disposable absorbent undergarment snuggly conforms to the wearer's torso.

The elastic strands can be coated with an adhesive. By adhesively coating each of the elastic strands, instead of slot coating a major portion of the inner surface of at least one of the first and second layers, softer moving webs 12 respectively, can be obtained. Wearers of disposable absorbent undergarments prefer a product that has a softer feel since it is more like underwear.

In other embodiments, the elastomeric nonwoven material comprises an elastomeric film sandwiched between two nonwoven facing layers. U.S. Pat. No. 7,803,244 to Siqueira et al., hereby incorporated by reference, discloses particular examples of elastomeric nonwoven composites suitable for use in the adjustable undergarment described herein.

It should be noted that the moving web 12 can be formed from a breathable or a non-breathable material. Desirably, the moving web 12 is formed from a breathable material or a material that is treated or processed to be breathable. Spunbond and bonded carded webs are two breathable materials that work well as moving web 12 in disposable absorbent undergarments. Bonded carded webs are produced and commercially sold by a variety of vendors. Other materials that can be used to form the moving web 12 include woven and non-woven materials formed from natural or synthetic fibers; polyolefins, such as polypropylene or polyethylene; thermoplastic films; as well as other materials known to those skilled in the art. A metallocene polypropylene works very well since it has a soft feel and can be easily ultrasonically bonded to itself.

The moving web 12 is first passed through a weakening station 20 at a first position A1. The weakening station 20 forms a line of weakness 17 in the moving web 12 to define a leading portion 14 of the web and a trailing portion of the web 16 connected at the line of weakness 17, as shown for example in FIG. 1. In a desirable embodiment, the weakening station 20 forms a cross-directional line for the line of weakness 17, which is preferably linear, in the web. The line of weakness 17 can extend across the entire cross-direction width of the web, or along only a portion thereof. In some embodiments, the line of weakness 17 can be tapered relative to the longitudinal axis, if desired. In addition, the line of weakness 17 can also be curved or a combination of geometric cut-out shapes.

In one desirable embodiment, the first weakening station 20 is configured as a perforator having a knife roll and an anvil roll. Alternatively, the weakening station 20 can be configured with a flex knife, laser, water jet, or other types of cutters known to those skilled in the art. In other alternative embodiments, the weakening station 20 can comprise a device for applying heat, thermal energy or ultrasonic energy to the web so as to weaken it at specific locations, or lines of weakness 17. In other preferred embodiments, the weakening station 20 can include a chemical applicator that applies various chemicals, including for example water, to the moving web 12 to weaken it at specific locations. In yet another alternative embodiment, the apparatus applies a speed differential to the moving web 12 so as to weaken the moving web 12. Of course, it should be understood that the weakening station 20 can also be configured from combinations of one or more of the above-referenced devices.

FIGS. 3 through FIGS. 7 depict exemplary embodiments for patterns for line of weakness 17. Of course, these examples are not exhaustive and only illustrate a few of the possible patterns. One skilled in the art may combine the illustrated patterns and utilize any other combination of patterns for a line of weakness 17 across the moving web 12. For each of these described embodiments, a line of weakness 17 extending in the machine direction extends in the transverse direction on the garment while a line of weakness 17 extending in the cross-machine direction extends in the longitudinal direction on the garment.

The line of weakness 17 may have a variety of configurations. In one embodiment, the line of weakness 17 includes a series of weakened areas 19, the series of weakened areas 19 having a combined length in a cross-machine direction at least 85% of a distance X between the waist end 22 and the leg cut-out end 21. In other words, the sum of the length of each of the weakened areas 19 of the line of weakness 17 in the cross-machine direction is equal to at least 85% of the distance X between the waist end 22 and the leg cut-out end 21. Having such a large percentage of the moving web 12 weakened allows for a product that is easier to break during use. In another embodiment, the series of weakened areas 19 have a combined length in a cross-machine direction at least 90% of a distance X between the waist end 22 and the leg cut-out end 21. In another embodiment, the series of weakened areas 19 have a combined length in a cross-machine direction at least 99% of a distance X between the waist end 22 and the leg cut-out end 21. For a product made as described herein, a line of weakness 19 extending in the machine direction extends in the transverse direction on the garment while a line of weakness 19 extending in the cross-machine direction extends in the longitudinal direction on the garment.

FIG. 3 illustrates one embodiment for a line of weakness 17 on a moving web 12. As illustrated, the line of weakness 17 includes a first region 40 at the waist end 22 having a first perforation pattern 41 and a second region 42 at the leg cut-out end 21 having a second perforation pattern 43. The line of weakness 17 includes a third region 44 extending between the first region 40 and second region 42, the third region 44 having a third perforation pattern 45. The third perforation pattern 45 is different than the first perforation pattern 41 and second perforation pattern 43.

In one embodiment, as illustrated in FIG. 3, the first perforation pattern 41 and second perforation pattern 43 have a first perforation length and the third perforation pattern 45 has a second perforation length, wherein the first perforation length is greater than the second perforation length. Having a longer perforation length at the waist end 22 and leg cut-out end 21 may allow for easier breakage of the line of weakness 17 on the product. In one particular, embodiment, the first perforation length is greater than about 10 mm.

In another embodiment, as illustrated in FIG. 4, the line of weakness 17 further includes a fourth region 50 in a middle portion 51 of the moving web 12, the fourth region 50 having at least one perforation 52 extending between 10% and 85% of the distance X between the waist end 22 and the leg cut-out end 21. As illustrated in FIG. 5, this fourth region 50 may include a plurality of perforations 53. Longer perforations residing in the middle portion 51 of the moving web 12 will allow for easier breakage of the web by a consumer during use.

In another embodiment, as illustrated in FIG. 6 the line of weakness 17 may further have a fifth region 60 extending between the fourth region 50 and the third region 45, the fifth region 60 having a fourth perforation pattern 62. This fourth perforation pattern 62 may have a perforation length of less than about 2 mm.

In an alternative embodiment, as illustrated in FIG. 7, the first perforation pattern 71 and second perforation pattern 72 may have a first perforation length and the third perforation pattern 74 may have a second perforation length, wherein the first perforation length is less than the second perforation length. Having a shorter perforation length at the waist end 22 and leg cut-out end 21 may allow for easier breakage of the line of weakness 17 on the product. In this particular embodiment, the first perforation length is less than about 2 mm and the second perforation length is greater than 2 mm. The embodiment in FIG. 7 may also include the various pattern changes in FIGS. 4-7.

After forming a line of weakness 17, the moving web 12 is passed through a fastener attachment station 30 at a second position A2. The fastener attachment station 30 attaches a fastener assembly 18 over the line of weakness 17 that defines a leading portion 14 of the moving web 12 and a trailing portion 16 of the moving web 12 together with a fastener assembly 18. The fastener assembly 18 will maintain the integrity of the moving web 12 as it continues through the remainder of the process.

Web panel stability is an important aspect to maintaining the integrity of the process. The stability of the moving web 12 can be maintained with use of a stability control module 15. The stability control module 15 may include vacuum, surface roughness and/or needle/clamp engagement or other suitable means known to those skilled in the art to ensure stability of the moving web 12 on the conveying means.

Desirably, the weakening station 20, stability control module 15, and the fastener assembly attachment station 30 are an integral component in a manufacturing process. For example, the stability control module 15 may be a vacuum drum. While the moving web 12 is conveyed on the vacuum drum, it passes through the weakening station 20 to create a line of weakness 17. The vacuum drum maintains the stability of the moving web 12 after it is weakened. Soon after weakening, the moving web 12 is conveyed on the vacuum drum through the fastener assembly attachment station 30. All of these components are incorporated into a single component in the manufacturing. In other embodiments, these steps may be included as separate components on a manufacturing line.

Various methods and apparatus for manufacturing absorbent garments and for applying fastener members thereto are disclosed in U.S. Pat. No. 6,730,188 entitled “Method and Apparatus For Assembling Refastenable Absorbent Garments,” U.S. Pat. No. 6,743,321 entitled “Method and Apparatus For Assembling Refastenable Absorbent Garments,” U.S. Pat. No. 6,686,626 entitled “Method and Apparatus For Assembling Refastenable Absorbent Garments,” U.S. Pat. No. 6,682,626 entitled “Method and Apparatus For Assembling Refastenable Absorbent Garments,” U.S. Pat. No. 6,712,922 entitled “Multiple Component Web,” U.S. Pat. No. 6,730,188 entitled “Method of Assembling Personal Care Absorbent Article,” U.S. Pat. No. 6,783,487, entitled “Pant-Type Personal Care Articles, and Methods of Making and Using Such Personal Care Articles,” U.S. Pat. No. 6,454,888 entitled “Methods of Changing Size of Pant-Type Personal Care Articles Outputted from a Manufacturing Process,” the entire disclosures of which are hereby incorporated by reference.

In one embodiment, illustrated in FIG. 1, the moving web 12 is used as a front body panel and bonded to a back body panel web 28 at side seams, wherein the rear body panel web 28 is positioned over the moving web 12 by folding a crotch portion 29 joining or bridging between the moving web 12 and back panel web 28 at a third location A3.

Referring to FIGS. 8-10, an adjustable pant-like disposable absorbent undergarment 100 having a longitudinal axis 111 made using the method described herein is shown. The adjustable pant-like disposable absorbent undergarment 100 is designed to absorb liquid, semi-solid and/or solid waste discharged from a human being. The adjustable pant-like disposable absorbent undergarment 100 is designed to absorb and/or retain one or more bodily discharges of waste material such as urine, perspiration, excrement, feces, menses, menstrual fluid, as well as other liquid and/or solid waste.

The adjustable pant-like disposable absorbent undergarment 100 includes a front body panel 112, a back body panel 114 and an absorbent assembly 116 secured to the front and back body panels, 112 and 114 respectively. The front and back body panels, 112 and 114 respectively, are joined together by a pair of seams 118 and 120 to form a waist opening 122 and a pair of leg openings 124 and 126.

The front body panel 112 includes a waist edge 128, a crotch edge 130 and a pair of side edges 132 and 134. In a three piece construction wherein an absorbent assembly 116 is secured between the front body panel 112 and the back body panel 114, the crotch edge 130 is well defined. In absorbent undergarments of a different construction, the crotch edge 130 can be an imaginary line transversely drawn between the pair of leg openings 124 and 126 at a location where one considers the front body panel 112 to end. The exact size and configuration of the front body panel 112 can vary to suit a wearer's particular needs. The front body panel 112 has a first side section 136, a middle section 138 and a second side section 140. The front body panel 112 contains a line or weakness between both the first side section 136 and the middle section 138 and the second side section 140 and the middle section 138 creating a first line of weakness 142 and a second line of weakness 144. The first side section 136 is aligned adjacent to the side edge 132 at its leading edge forming the seam 120 and the second side section 140 is aligned adjacent to the side edge 134 at its leading edge forming the seam 118. As depicted, the middle section 138 is centrally located and is bifurcated by the longitudinal axis 111 at a midpoint of the front body panel 112.

The first and second lines, 142 and 144, can be linear or non-linear in configuration. In FIGS. 9-10, the first and second line of weakness, 142 and 144, are shown having a linear or straight configuration. The first and second lines of weakness, 142 and 144, extend longitudinally from approximately the waist edge 128 down to approximately one of the leg openings, leg cut-outs 124 or 126. The first and second lines of weakness, 142 and 144, can be aligned parallel to the longitudinal axis 111 or be angled thereto. For example, the first and second lines of weakness, 142 and 144, can be tapered relative to the longitudinal axis 111, if desired. In addition, the lines of weakness, 142 and 144, can also be curved. Desirably, the lines of weakness 142 and 144 match the patterns found in FIGS. 3-7.

Still referring to FIGS. 8-10, each of the first and second lines of weakness, 142 and 144, extend from approximately the waist edge 128 of the front body panel 112 to one of the pair of leg openings 124 and 126. Another way of describing this is to say that the first and second lines of weakness, 142 and 144, extend from approximately the waist edge 128 of the front body panel 112 to approximately the crotch edge 130 of the front body panel 112.

Still referring to FIGS. 9-10, each of the first and second lines of weakness, 142 and 144, is shown being aligned parallel to one of the pair of seams 118 and 120. Such an arrangement provides for an aesthetically pleasing appearance to the front body panel 112.

Referring again to FIGS. 9-10, the front body panel 112 further includes a pair of fastener assemblies 168 and 170. Each fastener assembly 168 and 170 includes a first portion 172 and a second portion 174. The first portion 172 of the fastener assembly 168 can be permanently secured to the first side section 136 of the front body panel 112 and the first portion 172 of the other fastener assembly 170 can be permanently secured to the second side section 140 of the front body panel 112.

In the embodiment shown in FIGS. 9-10, the first portions 172 of the fastener assemblies 168 and 170 can be securely attached using an adhesive, heat, pressure, a combination of heat and pressure, an ultrasonic bond, a chemical bond or by other means known to those skilled in the art.

Each of the second portions 174 of the fastener assemblies 168 and 170 are depicted as extending almost the entire length of the lines of weakness 142 and 144. This means at least 50% of the distance between the waist edge 128 and the leg opening 124 are covered by the fastener assembly 168. Desirably, the first fastener assembly 168 and second fastener assembly 170 extend at least 65% of the distance between the waist edge 128 and the leg opening 124. More desirably, the first fastener assembly 168 and second fastener assembly 170 extend at least 75% of the distance between the waist edge 128 and the leg opening 124. Even more desirably, the first fastener assembly 168 and second fastener assembly 170 extend at least 85% of the distance between the waist edge 128 and the leg opening 124. Even more desirably, the first fastener assembly 168 and second fastener assembly 170 extend at least 95% of the distance between the waist edge 128 and the leg opening 124. The second portions 174 of the first fastener assembly 168 can bridge across the first line of weakness 142 and the second portion 174 of the other fastener assembly 170 can bridge across the second line of weakness 144. By extending substantially the entire length of and bridging the lines of weakness, 142 and 144, the fastener assemblies 168, 170 cover the lines of weakness 142 and 144, and provide a more underwear like appearance. The second portions 174 of the fastener assemblies 168 and 170 can be releasably attached to the middle section 138 of the front body panel 112.

Alternatively, it should be evident to those skilled in the art that the first portion 172 of each of the fastener assemblies 168 and 170 could be permanently attached to the middle section 138. In this embodiment, the second portions 174 of the fastener assemblies 168 and 170 can be releasably attached to the side sections 136 and 140 of the front body panel 112. In an alternative embodiment, both the first portion 172 and the second portion 174 may be releasably attached. The fastener assembly 168 may be constructed of a non-extensible or an elastomeric material.

Referring now to FIGS. 9-10, each of the second portions 174 of the fastener assemblies 168 and 170 has an inner surface 180 that contains a fastener 182. The fastener 182 can be a mechanical fastener 182. In FIGS. 9 and 10, the mechanical fastener 182 is shown as a plurality of fine hooks, such as VELCRO hooks. VELCRO is a trademark of Velcro USA, Inc. (Manchester, N.H.). The hooks are designed to easily engage and be removed from a material wherein the material has a loose weave pattern or the fibers forming the material will allow the hooks to be attached to them. The mating material is commonly referred to as the loop member of a hook and loop fastener. The middle section 138 of the front body panel 112 may be formed of such a material. When the hooks engage into the middle section 138, a secure but releasable fastener is formed. The hooks can be easily removed from the loop material by pulling the edge of the fastener outward away from the middle section 138. Alternatively, as illustrated in FIGS. 9 and 10, a fastening component 183 such as a loop material or loose fibers that may be placed anywhere onto the middle section 138 to facilitate the fastening component 183. Therefore, hook and loop fasteners are referred to as being releasable and can be fastened and released several times. In alternative embodiments, the mechanical fastener 182 constructed of hooks may be placed on to the middle section 138 of the front body panel 112. In this embodiment, the fastening component 183 constructed of a loop material or loose fibers would be placed on the fastener assemblies 168, 170. Both the mechanical fastener 182 and the fastening component 183 may be integral or separately attached.

Referring again to FIGS. 8-10, one can see that FIG. 8 depicts the pair of fastener assemblies 168 and 170 being securely fastened to the middle section 138 of the front body panel 112. If the wearer of the disposable absorbent undergarment 100 or a caregiver wishes to inspect the undergarment 100, he or she would open the pair of fastener assemblies 168 and 170 to the position shown in FIG. 9. If desired, a wearer or caregiver may break the lines of weakness 142, 144 to break the front body panel 112 and allow the product to be removed or adjusted. The disposable absorbent undergarment 100 can be removed from about the wearer's torso and be replaced by another undergarment. If the absorbent assembly 116 is still capable of accepting additional body fluid, the middle section 138 is moved back against the wearer's torso and the pair of fastener assemblies 168 and 170 is refastened to the middle section 138.

The horizontal distance to each of the first and second lines of weakness, 142 and 144, and corresponding first and second fastener assemblies, 168 and 170, as located relative to the pair of side seams 118 and 120 can also vary. A distance “D” is depicted in FIGS. 4-5 which represents the distance between each of the pair of seams 118 and 120 and the corresponding first and second lines of weakness, 142 and 144 respectively. One can increase the distance “D” by moving the first and second lines of weakness, 142 and 144 respectively, away from the pair of seams 118 and 120. When one increases the distance “D”, one may find that it is easier for the wearer of the disposable absorbent undergarment 100 to visually see and identify the first and second lines of weakness, 142 and 144, when the user looks down at the front body panel 112. In some embodiments, the first fastener assembly 168 and the second fastener assembly 170 are located near the midpoint between the side seams, 118 and 120, and the cross-sectional midpoint 111 of the front body panel 112. Desirably, the first fastener assembly 168 and second fastener assembly 170 are located between 30% and 70% of the distance between the side seam, 118 and 120, and the cross-sectional midpoint 111 of the front body panel 112. More desirably, the first fastener assembly 168 and second fastener assembly 170 are located between 35% and 45% of the distance between the side seams, 118 and 120, and the cross-sectional midpoint 111 of the front body panel 112. A manufacturer is free to vary the distance “D” to best suit the size and shape of a particular disposable absorbent undergarment 100 to make sure that the fastener is on the front of the product rather than the side of the product. If the lines of weakness 142, 144 are not parallel to the side seams 118, 120, the midpoint of the lines of weakness 142, 144 in the longitudinal direction may be used to measure “D”.

Placement near the midpoint between the side seam and the cross-sectional midpoint 111 of the front body panel 112 facilitates enhanced donning and removal when experiencing a reduced range of motion as well as locating the fastener in a location which is easier for the consumer to see.

The placement of the fastener assemblies 168 and 170 in the front of the product optimizes the ability of the user to fasten the product, while minimizing potential pop-opens during use due to movement of the legs of the user. The placement of the fasteners can be measured in both an unstretched (out of the bag) state or stretched (as in use).

It should be understood that various other embodiments, modifications, and equivalents to the embodiments of the absorbent article described herein which, after reading the description herein, may suggest themselves to those skilled in the art without departing from the scope and spirit of the present claims.

What is claimed is:

1. A pre-fastened adjustable pant-like disposable absorbent undergarment comprising:
a. a body panel having a waist end and a leg-cut-out end;
b. a fastener assembly attached to the body panel, wherein at least one side of the fastener assembly is releasably attached to the body panel;
c. a line of weakness under the fastener assembly defining a first section of the body panel and a second section of the body panel attached at the line of weakness, the fastener assembly bridging the first section of the body panel and the second section of the body panel; and
d. wherein the line of weakness comprises a series of weakened areas, the series of weakened areas having a combined length in a longitudinal direction at least 85% of a distance between the waist end and the leg cut-out end.

2. The pre-fastened adjustable pant-like disposable absorbent undergarment of claim 1 wherein the series of weakened areas having a combined length in the longitudinal direction at least 90% of the distance between the waist end and the leg cut-out end.

3. The pre-fastened adjustable pant-like disposable absorbent undergarment of claim 1 wherein the series of weakened areas having a combined length in the longitudinal direction at least 99% of the distance between the waist end and the leg cut-out end.

4. The pre-fastened adjustable pant-like disposable absorbent undergarment of claim 1 wherein the line of weakness comprises a series of intermittent perforations.

5. The pre-fastened adjustable pant-like disposable absorbent undergarment of claim 1 wherein the line of weakness comprises a scored web.

6. The pre-fastened adjustable pant-like disposable absorbent undergarment of claim 1 wherein the line of weakness comprises:
a. a first region at the waist end having a first perforation pattern;
b. a second region at the leg cut-out end having a second perforation pattern; and
c. a third region extending between the first region and second region, the third region having a third perforation pattern, wherein the third perforation pattern is different than the first and second perforation pattern.

7. The pre-fastened adjustable pant-like disposable absorbent undergarment of claim 6 wherein the first and second perforation pattern comprise a first perforation length and the third perforation pattern comprises a second perforation length, wherein the first perforation length is greater than the second perforation length.

8. The pre-fastened adjustable pant-like disposable absorbent undergarment of claim 7 wherein the first perforation length is greater than about 10 mm.

9. The pre-fastened adjustable pant-like disposable absorbent undergarment of claim 6 wherein the first and second perforation pattern comprise a first perforation length and the third perforation pattern comprises a second perforation length, wherein the first perforation length is less than the second perforation length.

10. The pre-fastened adjustable pant-like disposable absorbent undergarment of claim 9 wherein the first perforation length is less than about 2 mm and the second perforation length is greater than 2 mm.

11. The pre-fastened adjustable pant-like disposable absorbent undergarment of claim 6 wherein the line of weakness further comprises a fourth region in a middle of the body panel, the fourth region having at least one perforation extending between 10% and 85% of the distance between the waist end and the leg cut-out end.

12. The pre-fastened adjustable pant-like disposable absorbent undergarment of claim 11 wherein the line of weakness further comprises a fifth region extending between the fourth region and the third region, the fifth region having a fourth perforation pattern comprising a perforation length of less than about 2 mm.

13. A method of producing pre-fastened adjustable disposable absorbent garments, comprising:
a. providing a moving web for use as a body panel having a waist end and a leg-cut-out end;
b. weakening the moving web to form a line of weakness defining a leading portion of the moving web and a trailing portion of the moving web at the line of weakness, wherein the line of weakness comprises a series of weakened areas having a combined length in a cross-machine direction at least 85% of a distance between the waist end and the leg cut-out end; and
c. attaching a fastener assembly to the moving web such that the leading portion and trailing portion remain bridged together by the fastener assembly.

14. The method of claim 13 wherein the series of weakened areas comprise a combined length in the cross-machine direction at least 90% of the distance between the waist end and the leg cut-out end.

15. The method of claim 13 wherein the series of weakened areas having a combined length in the cross-machine direction at least 99% of the distance between the waist end and the leg cut-out end.

16. The method of claim 13 further comprising providing the moving web on a stability control module selected from vacuum, surface roughness, needle/clamp engagement and combinations thereof.

17. The method of claim 13 wherein the line of weakness comprises a series of intermittent perforations.

18. The method of claim 13 wherein the line of weakness comprises scoring the web to provide a weakened area.

19. The method of claim 13 wherein the line of weakness comprises:
a. a first region at the waist end having a first perforation pattern;
b. a second region at the leg cut-out end having a second perforation pattern; and
c. a third region extending between the first region and second region, the third region having a third perforation pattern, wherein the third perforation pattern is different than the first and second perforation pattern.

20. The method of claim 19 wherein the first and second perforation patterns comprise a first perforation length and the third perforation pattern comprises a second perforation length, wherein the first perforation length is greater than the second perforation length.

21. The method of claim 19 wherein the first perforation length is greater than about 10 mm.

22. The method of claim 19 wherein the first and second perforation patterns comprise a first perforation length and the third perforation pattern comprises a second perforation length, wherein the first perforation length is less than the second perforation length.

23. The method of claim 19 wherein the first perforation length is less than about 2 mm and the second perforation length is greater than 2 mm.

24. The method of claim 19 wherein the line of weakness further comprises a fourth region in a middle of the body panel, the fourth region having at least one perforation extending between 10% and 85% of the distance between the waist end and the leg cut-out end.

25. The method of claim 19 wherein the line of weakness further comprises a fifth region extending between the fourth region and the third region, the fifth region having a fourth perforation pattern comprising a perforation length of less than about 2 mm.
